(19) United States
(12) Patent Application Publication
(10) Pub. No.: US 2005/0222212 A1
(43) Pub. Date: Oct. 6, 2005

(54) Compounds and methods

(76) Inventors: Joseph P. Marino, King of Prussia, PA (US); Scott K. Thompson, King of Prussia, PA (US); Leslie M. Palmer, Collegeville, PA (US); Guang Yang, San Diego, CA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/509,214

(22) PCT Filed: Mar. 26, 2003
(86) PCT No.: PCT/US03/09276

§ 371 Date: Sep. 24, 2004

Related U.S. Application Data
(60) Provisional application No. 60/369,739, filed on Mar. 27, 2002.

(51) Int. Cl. A61K 31/4439; A61K 31/4196

(52) U.S. Cl. 514/341; 514/383

(57) ABSTRACT

Compounds of this invention are non-peptide, reversible inhibitors of bacterial methionine aminopeptidases, useful in treating bacterial infections.

### FIELD OF THE INVENTION

Compounds of this invention are non-peptide, reversible inhibitors of bacterial methionine aminopeptidases, useful as anti-bacterial agents.

### BACKGROUND OF THE INVENTION

Methionine aminopeptidases are ubiquitously distributed in all living organisms. They catalyze the removal of the initiator methionine from newly translated polypeptides using divalent metal ions as cofactors. Two distantly related MetAP enzymes, type 1 and type 2, are found in eukaryotes, which at least in yeast, are both required for normal growth; whereas only one single MetAP is found in eubacteria (type 1) and archaebacteria (type 2). The N-terminal extension region distinguishes the MetAPs in eukaryotes from those in procaryotes. A 64-amino acid sequence insertion (from residues 381 to 444 in hMetAP2) in the catalytic C-terminal domain distinguishes the MetAP-2 family from the MetAP-1 family. Despite the difference in the gene structure, all MetAP enzymes appear to share a highly conserved catalytic scaffold termed “pita-bread” fold (Bazan, et al. (1994) Proc. Natl. Acad. Sci. U.S.A. 91, 2473), which contains six strictly conserved residues implicated in the coordination of the metal cofactors.

N-terminal methionine removal in bacteria is a two-step process requiring first the removal on the N-formyl group by polypeptide deformylase followed by cleavage of the N-terminal methionine when the adjacent amino acid is small (e.g., Ala, Pro, Ser, Thr, Gly, Cys, and Val). Both of these steps are essential for cell viability. Failure to remove the N-terminal methionine can lead to inactive enzymes (e.g., glutamine phosphoribosylpyrophosphate amidotransferase and N-terminal nucleophile hydrolases). Therefore, inhibition of MetAP may have a wide-ranging effect inhibiting the action of essential enzymes involved in varied cellular processes.

MetAP is an attractive antibacterial target as this enzyme has been demonstrated to be essential for bacterial growth in vitro (Chang, et al. (1989) J. Bacteriol. 171, 4071, and Miller et al. (1989) J. Bacteriol. 171, 5215.); and is universally conserved in prokaryotes. This indicates that inhibitors directed against this target will be broad-spectrum agents and will kill bacteria. Further, we have shown that this gene is transcribed in thigh lesion and pyelonephritis models of infection with S. aureus as well as both early and late in murine respiratory tract infection with S. pneumoniae indicating the importance of this process in infection.

### SUMMARY OF THE INVENTION

In one aspect, the present invention is to a method of treating bacterial infections in mammals by administering to a mammal in need of such treatment, a compound of formula (IA), or a pharmaceutically acceptable salt or solvate thereof 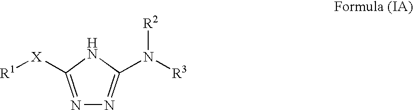
wherein,
X is S or O;
R¹ is optionally substituted C₁₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl;
R² is optionally substituted C₂₋₆alkyl, C₃₋₆alkenyl, C₃₋₆alkanyl, optionally substituted Ar—C₀₋₆alkyl, optionally substituted Het-C₀₋₆alkyl, C₃₋₇cycloalkyl-C₀₋₆alkyl;
R³ is H, optionally substituted C₁₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl, C₀₋₆alkyl-C(O)X′AB, C₀₋₆alkyl-S(O)₂X′AB, C₀₋₆alkyl-X′AB, wherein X′ is O, S, C or N; A and B are independently H, optionally substituted C₁₋₆alkyl, C₃₋₆alkenyl, C₃₋₆alkynyl, optionally substituted Ar—C₀₋₆alkyl, optionally substituted Het-C₀₋₆alkyl, C₃₋₇cycloalkyl-C₀₋₆alkyl, or A or B are independently absent.

In another aspect, the present invention is to a method of inhibiting bacterial MetAP in the treatment of bacterial infections, all in mammals, preferably humans, comprising administering to such mammal in need thereof, a compound of formula (IA), or a pharmaceutically active salt or solvate thereof.

### DETAILED DESCRIPTION OF THE INVENTION

It has now been discovered that substituted 1,2,4-triazoles of formulae (I) and (IA) are inhibitors of bacterial MetAP. It has also now been discovered that selective inhibition of bacterial MetAP mechanisms by treatment with the inhibitors of formula (IA), or a pharmaceutically acceptable salt or solvate thereof, represents a novel therapeutic and preventative approach to the treatment of a variety of bacterial infections, including, but not limited to infections caused by Staphylococcus aureus, Streptococcus pneumoniae, and haemophilus influenzae.

The term “C₁₋₆alkyl” as used herein at all occurrences means a substituted and unsubstituted, straight or branched chain radical of 1 to 6 carbon atoms, unless the chain length is limited thereto, including, but not limited to methyl, ethyl, n-propyl, isopropyl, n-butyl, isobutyl and t-butyl, pentyl, n-pentyl, isopentyl, neopentyl and hexyl and the simple aliphatic isomers thereof. Any C₁₋₆alkyl group may be optionally substituted independently by one or more of OR⁴, R⁴, NR⁴R⁵. C₀alkyl means that no alkyl group is present in the moiety. Thus, Ar—C₀alkyl is equivalent to Ar.

As used herein at all occurrences, substituents R⁴, R⁵, and R⁶ are independently defined as C₂₋₆alkyl, C₃₋₆alkenyl, C₃₋₆alkynyl, Ar—C₀₋₆alkyl, Het-C₀₋₆alkyl, or C₃₋₇cycloalkyl-C₀₋₆alkyl.

The term “C₃₋₇cycloalkyl” as used herein at all occurrences means substituted or unsubstituted cyclic radicals having 3 to 7 carbons, including but not limited to cyclopropyl, cyclopentyl, cyclohexyl and cycloheptyl radicals.

The term “C₃₋₆alkenyl” as used herein at all occurrences means an alkyl group of 3 to 6 carbons wherein a carbon-carbon single bond is replaced by a carbon-carbon double bond. C₃₋₆alkenyl includes 1-propene, 2-propene, 1-butene, 2-butene, isobutene and the several isomeric pentenes and hexenes. Both cis and trans isomers are included within the scope of this invention. Any C₃₋₆alkenyl group may be optionally substituted independently by one or more of Ph-C₀₋₆alkyl, Het′-C₀₋₆ alkyl, C₁₋₆alkyl, C₁₋₆alkoxy, C₁₋₆mercaptyl, Ph-C₀₋₆alkoxy, Het′-C₀₋₆alkoxy, OH, NR⁴R⁵, Het′-S—C₀₋₆alkyl, (CH₂)₁₋₆OH, (CH₂)₁₋₆NR⁴R⁵, O(CH₂)₁₋₆NR⁴R⁵, (CH₂)₀₋₆CO₂R⁶, O(CH₂)₁₋₆CO₂ R⁶, (CH₂)₁₋₆SO₂, CF₃, OCF₃ or halogen.

The term “C₃₋₆alkynyl” as used herein at all occurrences means an alkyl group of 3 to 6 carbons wherein one carbon-carbon single bond is replaced by a carbon-carbon triple bond. C₃₋₆ alkynyl includes 1-propyne, 2-propyne, 1-butyne, 2-butyne, 3-butyne and the simple isomers of pentyne and hexyne.

The terms “Ar” or “aryl” as used herein interchangeably at all occurrences mean phenyl and naphthyl, optionally substituted by one or more of Ph-C₀₋₆alkyl, Het′-C₀₋₆ alkyl, C₁₋₆alkyl, C₁₋₆alkoxy, C₁₋₆mercaptyl, Ph-C₀₋₆alkoxy, Het′-C₀₋₆alkoxy, OH, NR⁴R⁵, Het′-S—C₀₋₆alkyl, (CH₂)₁₋₆OH, (CH₂)₁₋₆NR⁴R⁵, O(CH₂)₁₋₆NR⁴R⁵, (CH₂)₀₋₆CO₂R⁶, O(CH₂)₁₋₆CO₂ R⁶, (CH₂)₁₋₆SO₂, CF₃, OCF₃ or halogen; in addition, Ph may be optionally substituted with one or more of C₁₋₆alkyl, C₁₋₆alkoxy, OH, (CH₂)₁₋₆NR⁴R⁵, O(CH₂)₁₋₆NR⁴R⁵, CO₂R⁶, CF₃, or halogen; Het′ is defined as for Het, and may be optionally substituted by one or more of C₁₋₆alkyl, C₁₋₆alkoxy, OH, (CH₂)₁₆NR⁴R⁵, O(CH₂)₁₋₆NR⁴R⁵, CO₂R⁶, CF₃, or halogen; or two C₁₋₆alkyl or C₁₋₆alkoxy groups may be combined to form a 5-7 membered, saturated or unsaturated ring, fused onto the Ar ring.

Suitably, for compounds of formula (I), when Ar is substituted by Ph or Het′, then Ph or Het′ are substituted with one or more of C₂₋₆alkyl, C₁₋₆alkoxy, (CH₂)₁₋₆NR⁴R⁵, O(CH₂)₁₋₆NR⁴R⁵, CO₂R⁶, CF₃ or halogen.

The terms “Het” or “heterocyclic” as used herein interchangeably at all occurrences, mean a stable 5- to 7-membered monocyclic, a stable 7- to 10-membered bicyclic, or a stable 11- to 18-membered tricyclic heterocyclic ring, all of which are either saturated or unsaturated, and consist of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure.

It will be understood that Het may be optionally substituted with one or more of Ph-C₀₋₆alkyl, Het′-C₀₋₆ alkyl, C₁₋₆alkyl, C₁₋₆alkoxy, C₁₋₆mercaptyl, Ph-C₀₋₆alkoxy, Het′-C₀₋₆alkoxy, OH, NR⁴R⁵, Het′-S—C₀₋₆alkyl, (CH₂)₁₋₆OH, (CH₂)₁₋₆NR⁴R⁵, O(CH₂)₁₋₆NR⁴R⁵, (CH₂)₀₋₆CO₂R⁶, O(CH₂)₁₋₆CO₂ R⁶, (CH₂)₁₋₆SO₂, CF₃, OCF₃, CN, or halogen; Ph may be optionally substituted with one or more of C₁₋₆alkyl, C₁₋₆alkoxy, OH, (CH₂)₁₋₆NR⁴R⁵, O(CH₂)₁₋₆NR⁴R⁵, CO₂R⁶, CF₃, or halogen; and two C₁₋₆alkyl or C₁₋₆alkoxy groups may be combined to form a 5-7 membered ring, saturated or unsaturated, fused onto the Het ring. Preferred optional substituents on Het are C₁₋₆alkyl, C₁₋₆alkoxy, C₁₋₆mercaptyl, halogen, CF₃, OCF₃, CN, or NR⁴R⁵.

Het′ is defined as for Het and may be optionally substituted by one or more of C₁₋₆alkyl, C₁₋₆alkoxy, OH, (CH₂)₁₋₆NR⁴R⁵, O(CH₂)₁₋₆NR⁴R⁵, CO₂R⁶, CF₃, or halogen.

Examples of such heterocycles include, but are not limited to piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, benzofuranyl, benzothiophenyl, thiamorpholinyl sulfoxide, thiamorpholinyl sulfone, and oxadiazolyl, triazolyl, thiadiazolyl, oxadiazolyl, isoxazolyl, isothiazolyl, imidazolyl, pyridazinyl, pyrimidinyl and triazinyl which are available by routine chemical synthesis and are stable.

Compounds of this invention of formula (I), do not include compounds wherein R² is optionally substituted Het-C₀alkyl, and Het is indole, benzofuran, benzothiophene, benzisoxazole, benzothiozole or benzopyrazole, and the optional substituent is —(CH₂)₂NR⁴R⁵. The following compounds of this invention are known: 3-(4-methyl-anilino)-5-benzylthio-1,2,4-triazole, 3-(2-methyl-anilino)-5-benzylthio-1,2,4-triazole, 3-(4-methoxy-anilino)-5-benzylthio-1,2,4-triazole, 3-(2-methoxy-anilino)-5-benzylthio-1,2,4-triazole, or 3-ethyl-3-anilino-5-benzylthio-1,2,4-triazole. Fromm et al., Justus Liebigs Ann. Chem., 437 1924, 113. A compound of formula (I) wherein R¹ is benzyl, R² is phenyl and R³ is hydrogen is known.

Suitably, when moieties R¹, R², or R³ are either optionally substituted Ar—C₀₋₆alkyl or optionally substituted Het-C₀₋₆alkyl, the moiety may be attached to the triazole substituent through the aromatic ring or through the alkyl chain.

Further, it will be understood that when a moiety is “optionally substituted” the moiety may have one or more optional substituents, each optional substituent being independently selected.

The terms “hetero” or “heteroatomn” as used herein interchangeably at all occurrences mean oxygen, nitrogen and sulfur.

The terms “halo” or “halogen” as used herein interchangeably at all occurrences mean F, Cl, Br, and I.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that C is 0.

Suitably X is sulfur or oxygen. Preferably X is sulfur.

Suitably, R¹ is optionally substituted C₂₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl. Preferably R′ is optionally substituted Ar—C₀₋₆alkyl or optionally substituted Het-C₀₋₆alkyl. More preferably R¹ is optionally substituted Ar—C₁alkyl or optionally substituted Het-C₁alkyl. Most preferably R¹ is optionally substituted benzyl, optionally substituted methylfuran or optionally substituted methylthiophene. Preferably, when R¹ is Het-C₁alkyl, the alkyl chain is directly attached to moiety X.

Suitably, R² is optionally substituted C₁₋₆alkyl, C₃₋₆alkenyl, C₃₋₆alkynyl, optionally substituted Ar—C₀₋₆alkyl, optionally substituted Het-C₀₋₆alkyl, C₃₋₇cycloalkyl-C₀₋₆alkyl. Preferably, R² is optionally substituted Ar—C₀₋₆alkyl. More preferably R² is optionally substituted Ar—C₀alkyl.

Suitably, R³ is H, optionally substituted C₁₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl, C₀₋₆alkyl-C(O)X′AB, C₀₋₆alkyl-S(O)₂X′AB, C₀₋₆alkyl-X′AB, wherein X′ is O, S, C or N; A and B are independently H, optionally substituted C₁₋₆alkyl, C₃₋₆alkenyl, C₃₋₆alkynyl, optionally substituted Ar—CO₀₋₆alkyl, optionally substituted Het-C₀₋₆alkyl, C₃₋₇cycloalkyl-C₀₋₆alkyl, or A or B are independently absent.

A preferred compound of this invention is a compound of formula (IB):

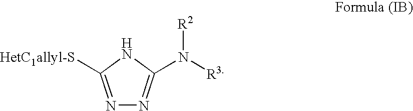

Suitably, pharmaceutically acceptable salts of formulae (IA) and (IB) include, but are not limited to, salts with inorganic acids such as hydrochloride, sulfate, phosphate, diphosphate, hydrobromide, and nitrate, or salts with an organic acid such as malate, maleate, fumarate, tartrate, succinate, citrate, acetate, lactate, methanesulfonate, p-toluenesulfonate, palmitate, salicylate, and stearate.

The compounds of the present invention may contain one or more asymmetric carbon atoms and may exist in racemic and optically active forms. The stereocenters may be (R), (S) or any combination of R and S configuration, for example, (R,R), (R,S), (S,S) or (S,R). All of these compounds are within the scope of the present invention.

All compounds of formula (IA) specifically named herein are considered to be part of the invention disclosed herein. Among the compounds of the invention of formula (IA) are the following compounds:
3-anilino-5-benzylthio-1,2,4-triazole;
3-anilino-5-(thiophen-2-ylmethylthio)-1,2,4-triazole;
3-anilino-5-(3,5-dimethyl-benzylthio)-1,2,4-triazole;
3-(4-methyl-anilino)-5-(pyridin-4-ylmethylthio)-1,2,4-triazole;
3-(2-methyl-anilino)-5-benzylthio-1,2,4-triazole;
3-(4-methyl-anilino)-5-(pyridin-2-ylmethylthio)-1,2,4-triazole;
3-(4-chloro-anilino)-5-(thiophen-2-ylmethylthio)-1,2,4-triazole;
3-(2-methyl-anilino)-5-(thiophen-2-ylmethylthio)-1,2,4-triazole;
3-(4-methoxy-anilino)-5-(thiophen-2-ylmethylthio)-1,2,4-triazole;
3-(4-methoxy-anilino)-5-(pyridin-4-ylmethylthio)-1,2,4-triazole;
3-(4-methoxy-anilino)-5-(2-methyl-benzylthio)-1,2,4-triazole;
3-(3,4-dimethoxy-anilino)-5-(3-methoxy-benzylthio)-1,2,4-triazole;
3-(3,4-dimethoxy-anilino)-5-(pyridin-2-ylmethylthio)-1,2,4-triazole;
[5-(benzylthio)-1H-[1,2,4]triazol-3-yl]-pyridin-3-yl-amine;
[5-(3-methoxybenzylthio)-1H-[1,2,4]triazol-3-yl]-pyridin-3-yl-amine;
[5-(2-fluoro-benzylthio)-1H-[1,2,4]triazol-3-yl]-pyridin-3-yl-amine;
[5-(2-methyl-benzylthio)-1H-[1,2,4]triazol-3-yl]-pyridin-3-yl-amine;
[5-(3,4-difluoro-benzylthio)-1H-[1,2,4]triazol-3-yl]-pyridin-3-yl-amine;
[5-(2-methoxy-benzylthio)-1H-[1,2,4]triazol-3-yl]-pyridin-3-yl-amine;
[5-(2-methyl-thiazol-4-ylmethylthio)-1H-[1,2,4]triazol-3-yl]-pyridin-3-yl-amine;
3-(2,4-dimethoxy-anilino)-5-(5-methyl-isoxazol-3-ylmethylthio)-1,2,4-triazole;
3-(2-methyl-4-methoxy-anilino)-5-(2-fluoro-benzylthio)-1,2,4-triazole;
3-(2-methyl-4-methoxy-anilino)-5-(5-methyl-isoxazol-3-ylmethylthio)-1,2,4-triazole;
3-(2-methyl-4-methoxy-anilino)-5-(pyridin-2-ylmethylthio)-1,2,4-triazole;
3-(3-methyl-anilino)-5-benzylthio-1,2,4-triazole;
3-(2,6-dimethyl-anilino)-5-(4-fluoro-benzylthio)-1,2,4-triazole;
3-(2,6-dimethyl-anilino)-5-(3,4-difluoro-benzylthio)-1,2,4-triazole;
3-(2,6-dimethyl-anilino)-5-(2-methyl-2-butenylthio)-1,2,4-triazole;
3-(2,6-dimethyl-anilino)-5-(2-fluoro-benzylthio)-1,2,4-triazole;
3-(2,6-dimethyl-anilino)-5-benzylthio-1,2,4-triazole;
3-(2,6-dimethyl-anilino)-5-(2-methyl-benzylthio)-1,2,4-triazole;
3-(2-ethyl-anilino)-5-(thiophen-2-ylmethylthio)-1,2,4-triazole;
3-(3-methyl-anilino)-5-(thiophen-2-ylmethylthio)-1,2,4-triazole;
3-(2-phenyl-anilino)-5-(thiophen-2-ylmethylthio)-1,2,4-triazole;
3-(2,4-dimethoxy-anilino)-5-(thiophen-2-ylmethylthio)-1,2,4-triazole;
[5-(thiophen-2-ylthio)-1H-[1,2,4]triazol-3-yl]-pyridin-3-yl-amine;
3-(2-methyl-4-methoxy-anilino)-5-(thiophen-2-ylmethylthio)-1,2,4-triazole;
3-(4-hydroxy-anilino)-5-benzylthio-1,2,4-triazole;
3-(2-methoxy-anilino)-5-(furan-2-ylmethylthio)-1,2,4-triazole;
3-anilino-5-(5-methyl-thiophen-2-ylmethylthio)-1,2,4-triazole;
3-(2-methyl-anilino)-5-(5-bromo-thiophen-2-ylmethylthio)-1,2,4-triazole;
3-(2-methoxy-anilino)-5-(5-chloro-thiophen-2-ylmethylthio)-1,2,4-triazole;
3-(3-methyl-anilino)-5-(3chloro-thiophen-2-ylmethylthio)-1,2,4triazole;
3-anilino-5-(5-chloro-thiophen-2-ylmethylthio)-1,2,4-triazole;
3-(3-methyl-anilino)-5-(furan-2-ylmethylthio)-1,2,4-triazole;
3-(2-Hydroxy-anilino)-5-benzylthio-1,2,4-triazole;
3-(3-methoxy-anilino)-5-(thiophen-2-ylmethylthio)-1,2,4-triazole;
3-(sec-butyl-anilino)-5-(furan-2-ylmethylthio)-1,2,4-triazole;
3-(3-methoxy-anilino)-5-(furan-2-ylmethylthio)-1,2,4-triazole;
3-(4-methoxy-anilino)-5-(furan-2-ylmethylthio)-1,2,4-triazole; and
3-(5-Benzyl-1H-[1,2,4]-triazole-3-yl sulfanyl)-propionic acid methyl ester.
   Methods of Preparation

Compounds of the formulae (IA) and (IB) wherein X is S and R³ is H, were prepared by methods analogous to those described in Scheme 1.

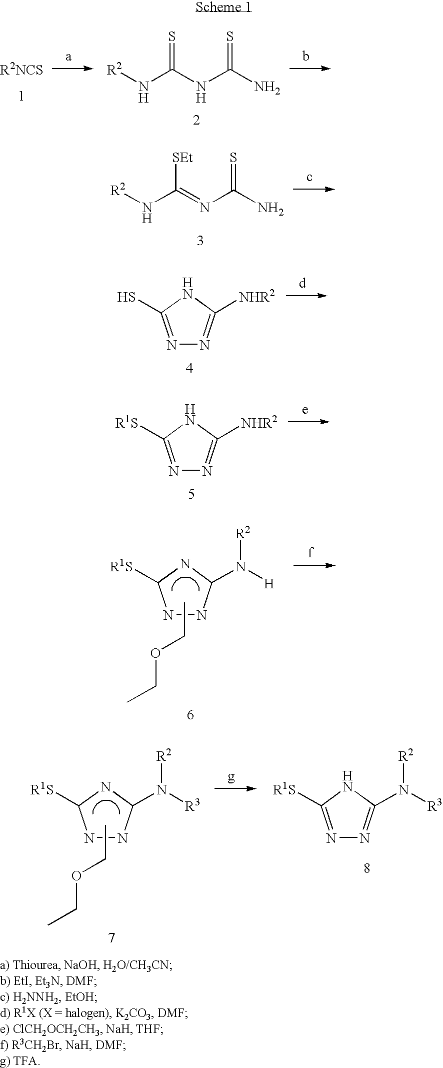

An isothiocyanate (such as phenyl isothiocyanate) (1-Scheme 1) was treated with thiourea and sodium hydroxide in acetonitrile/water to provide 2-Scheme 1, which was treated with iodoethane and triethylamine in DMF to afford 3-Scheme 1. Treatment of 3-Scheme 1 with hydrazine in ethanol provided 4-Scheme 1, which was treated with an alkyl halide (such as benzyl bromide or 4-chlorobenzyl chloride) and potassium carbonate in DMF to give Scheme 1. Triazole 5-Scheme 1 is protected as the methoxy methylethyl ether to afford 6-Scheme 1. Alkylation of 6-Scheme 1 with an alkyl halide (such as methyliodide, ethyliodide, i-isobutyl iodide, n-propyliodide, butyliodide, allylbromide, benzylbromide, and methyl bromoacetate) afforded the desired tertiary amine 7-Scheme 1. Deprotection of the MOM-ether 7-Scheme 1 with trifluoroacetic acid (TFA) provided the desired product 8-Scheme 1.

Compounds of the formulae (IA) and (IB) wherein X is O may be prepared by methods analogous to those described in Scheme 2.

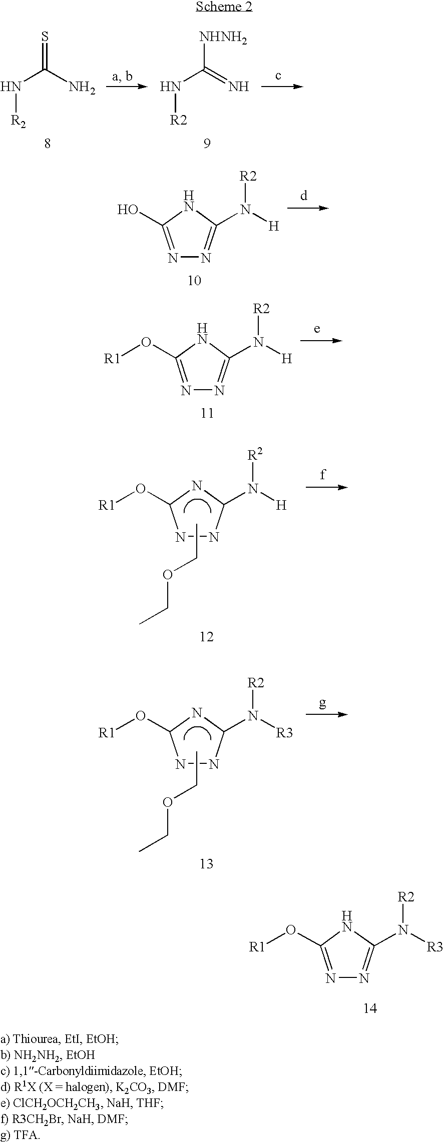

A thiourea (such as phenylthiourea) (8-Scheme 2) may be treated with ethyl iodide and refluxed in EtOH, and the resulting S-ethyl thiourea is then heated in the presence of hydrazine to provide 9-Scheme 2. The hydrazine 9-Scheme 2 is treated with carbonyldiimidazole and heated to afford 10-Scheme 2. Treatment of 10-Scheme 2 with an alkyl halide (such as benzyl bromide or 4-chlorobenzyl chloride) and potassium carbonate in DMF gives 11-Scheme 2. Triazole 11-Scheme 2 is protected as the methoxy methylethyl ether to afford 12-Scheme 2. Alkylation of 12-Scheme 2 with an alkyl halide (such as methyliodide, ethyliodide, i-isobutyl iodide, n-propyliodide, butyliodide, allylbromide, benzylbromide, and methyl bromoacetate) affords the desired tertiary amine 13-Scheme 2. Deprotection of the MOM-ether 13-Scheme 2 with trifluoroacetic acid (TFA) provides the desired product 14-Scheme 2.

Formulation of Pharmaceutical Compositions

The pharmaceutically effective compounds of this invention (and the pharmaceutically acceptable salts thereof) are administered in conventional dosage forms prepared by combining a compound of this invention (“active ingredient”) in an amount sufficient to treat bacterial infections with standard pharmaceutical carriers or diluents according to conventional procedures well known in the art. These procedures may involve mixing, granulating and compressing or dissolving the ingredients as appropriate to the desired preparation.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl monostearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1000 mg. When a liquid carrier is used, the preparation will be in the form of a syrup, emulsion, soft gelatin capsule, sterile injectable liquid such as an ampule or nonaqueous liquid suspension.

The active ingredient may also be administered topically to a mammal in need of treatment or prophylaxis of MetAP-mediated disease states. The amount of active ingredient required for therapeutic effect on topical administration will, of course, vary with the compound chosen, the nature and severity of the disease state being treated and the mammal undergoing treatment, and is ultimately at the discretion of the physician. A suitable dose of an active ingredient is 1.5 mg to 500 mg for topical administration, the most preferred dosage being 1 mg to 100 mg, for example 5 to 25 mg administered two or three times daily.

By topical administration is meant non-systemic administration and includes the application of the active ingredient externally to the epidermis, to the buccal cavity and instillation of such a compound into the ear, eye and nose, and where the compound does not significantly enter the blood stream. By systemic administration is meant oral, intravenous, intraperitoneal and intramuscular administration.

While it is possible for an active ingredient to be administered alone as the raw chemical, it is preferable to present it as a pharmaceutical formulation. The active ingredient may comprise, for topical administration, from 0.001% to 10% w/w, e.g. from 1% to 2% by weight of the formulation although it may comprise as much as 10% w/w but preferably not in excess of 5% w/w and more preferably from 0.1% to 1% w/w of the formulation.

The topical formulations of the present invention, both for veterinary and for human medical use, comprise an active ingredient together with one or more acceptable carrier(s) therefor and optionally any other therapeutic ingredient(s). The carrier(s) must be ‘acceptable’ in the sense of being compatible with the other ingredients of the formulation and not deleterious to the recipient thereof.

Formulations suitable for topical administration include liquid or semi-liquid preparations suitable for penetration through the skin to the site of inflammation such as liniments, lotions, creams, ointments or pastes, and drops suitable for administration to the eye, ear or nose.

Drops according to the present invention may comprise sterile aqueous or oily solutions or suspensions and may be prepared by dissolving the active ingredient in a suitable aqueous or alcoholic solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol. The formulation may incorporate any suitable surface-active agent such as an anionic, cationic or non-ionic surfactant such as esters or polyoxyethylene derivatives thereof. Suspending agents such as natural gums, cellulose derivatives or inorganic materials such as silicaceous silicas, and other ingredients such as lanolin, may also be included.

The active ingredient may also be administered by inhalation. By “inhalation” is meant intranasal and oral inhalation administration. Appropriate dosage forms for such administration, such as an aerosol formulation or a metered dose inhaler, may be prepared by conventional techniques. The daily dosage amount of the active ingredient administered by inhalation is from about 0.1 mg to about 100 mg per day, preferably about 1 mg to about 10 mg per day.

In one aspect, this invention relates to a method of treating bacterial infections in mammals, preferably humans, which comprises administering to such mammal an effective amount of a MetAP inhibitor, in particular, a compound of this invention.

By the term “treating” is meant either prophylactic or therapeutic therapy. Such compound can be administered to such mammal in a conventional dosage form prepared by combining the compound of this invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. The compound is administered to a mammal in need of treatment for bacterial infections, in an amount sufficient to decrease symptoms associated with these disease states. The route of administration may be oral or parenteral.

The term parenteral as used herein includes intravenous, intramuscular, subcutaneous, intra-rectal, intravaginal or intraperitoneal administration. The subcutaneous and intramuscular forms of parenteral administration are generally preferred. The daily parenteral dosage regimen will preferably be from about 30 mg to about 300 mg per day of active ingredient. The daily oral dosage regimen will preferably be from about 100 mg to about 2000 mg per day of active ingredient.

It will be recognized by one of skill in the art that the optimal quantity and spacing of individual dosages of a compound of this invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the compound given per day for a defined number of days, can be ascertained by those skilled in the art using conventional course of treatment determination tests.

### EXAMPLES

The invention will now be described by reference to the following examples which are merely illustrative and are not to be construed as a limitation of the scope of the present invention. In the Examples, proton NMR spectra were performed upon a Bruker 400 MHz NMR spectrometer, unless otherwise indicated.

### Example 1

### Preparation of 3-anilino-5-benzylthio-1,2,4-triazole

### a) 1-Phenyl-2,4-dithiobiuret

To a stirring solution of NaOH (0.52 g, 13.1 mmol) in 60 mL of 10% H₂O:CH₃CN was added thiourea (1.0 g, 13.1 mmol). The resulting suspension was heated to 40° C. for 20 min. and then cooled to RT. To this mixture was added phenylisothiocyanate (1.5 ml, 13.1 mmol), and the clear yellow solution was stirred overnight. After stirring for 12 h, 1 N HCl was added until a white precipitate formed. The precipitate was filtered, washed with H₂O, and dried under vacuum to produce the title compound as a light yellow powder (0.78 g, 30%). ¹H-NMR (400 MHz, d6-DMSO) δ 7.25 (t, 2H, J=7.3 Hz), 7.40 (t, 2H, J=7.9 Hz), 7.56 (d, 1H, J=7.9 Hz), 9.13-9.29 (broad singlet, 1H), and 10.26-10.79 (broad singlet, 2H).

### b) 2-Ethyl-1-phenyl-2-isodithiobiuret

To a stirring solution of the compound of Example 1(a) (150 mg, 0.70 mmol) in 4 mL of DMF was added triethylamine (57 uL, 0.70 mmol). The resulting yellow/green solution was stirred for 10 min at RT. To this solution was added ethyl iodide (100 uL, 0.70 mmol), and the reaction mixture was stirred for 2 h at RT. The yellow solution was poured into 20 mL of H₂O and extracted four times with EtOAc. The organic extracts were dried over Na₂SO₄, filtered, concentrated, and the crude residue was subjected to column chromatography (silica gel; ethyl acetate/hexane) to afford the title compound as a white crystalline solid (108 mg, 64%). ¹H-NMR (400 MHz, d6-DMSO) δ 1.22 (t, 3H, J=7.2 Hz), 2.96 (quartet, 2H, J=7.2 Hz), 6.85 (d, 1H, J=7.6 Hz), 7.16 (t, 1H, J=7.2 Hz), 7.29-7.41 (m, 3H), 8.27 (broad singlet, 1H), 9.89 (broad singlet, 1H), and 10.99 (broad singlet, 1H).

### c) 3-anilino-5-mercapto-1,2,4-triazole

To a stirring solution of the compound of Example 1(b) in 2 mL of EtOH was added 50 uL of anhydrous hydrazine. The reaction mixture was heated at 80° C. for 1 h, concentrated to dryness, and then purified by preparative HPLC to yield the title compound as a white solid (30 mg, 37%). MS (ESI) 190.90 (M−H)⁺.

### d) 3-anilino-5-benzylthio-1,2,4-triazole

To a stirring solution of the compound of Example 1(c) (23 mg, 0.12 mmol) in 1.2 mL of DMF was added K₂CO₃ (17 mg, 0.12 mmol), followed by benzyl bromide (20 mg, 0.12 mmol). The mixture was stirred overnight, filtered, and purified by preparative HPLC to afford the title compound as a white solid (30 mg, 70%). ¹H-NMR (400 MHz, d6-DMSO) δ 9.30 (broad singlet, 1H), 7.47 (d, 2H, J=8.1 Hz), 7.39 (d, 2H, J=7.3 Hz), 7.31 (t, 2H, J=7.3 Hz), 7.23 (quartet, 3H, J=7.3 Hz), 6.82 (t, 1H, J=7.3 Hz), and 4.3 (s, 2H).

### Example 2

### Preparation of 3-anilino-5-(thiophen-2-ylmethylthio)-1,2,4-triazole

Following the procedure of Example 1(a)-1(d), except 2-chloromethylthiophene was substituted for benzyl bromide in step 1(d), the title compound was prepared as a white solid. MS (ESI) 289.0 (M+H)⁺.

### Example 3

### Preparation of 3-(4-methyl-anilino)-5-(pyridin-4-ylmethylthio)-1,2,4-triazole

Following the procedure of Example 1(a)-1(d), except p-tolyl isothiocyanate was substituted for phenylisothiocyanate in step 1(a) and 4-(chloromethyl)pyridine was substituted for benzyl bromide in step 1(d), the title compound was prepared as a white solid. MS (ESI) 298.2 (M+H)⁺.

### Example 4

### Preparation of 3-(4-methyl-anilino)-5-(pyridin-2-ylmethylthio)-1,2,4-triazole

Following the procedure of Example 1(a)-1(d), except p-tolyl isothiocyanate was substituted for phenylisothiocyanate in step 1(a) and 2-(chloromethyl)pyridine was substituted for benzyl bromide in step 1(d), the title compound was prepared as a white solid. MS (ESI) 298.2 (M+H)⁺.

### Example 5

### Preparation of 3-(4-methoxy-anilino)-5-(pyridin-4-ylmethylthio)-112,4-triazole

Following the procedure of Example 1(a)-1(d), except p-methoxyphenyl isothiocyanate was substituted for phenylisothiocyanate in step 1(a) and 4-(chloromethyl)pyridine was substituted for benzyl bromide in step 1(d), the title compound was prepared as a white solid. MS (ESI) 314.2 (M+H)⁺.

### Example 6

### Preparation of 3-anilino-5-(3,5-dimethyl-benzylthio)-1,2,4-triazole

Following the procedure of Example 1(a)-1(d), except 3,5-dimethylbenzyl bromide was substituted for benzyl bromide in step 1(d), the title compound was prepared as a white solid. MS (ESI) 311.4 (M+H)⁺.

### Example 7

### Preparation of 3-(2-methyl-anilino)-5-(thiophen-2-ylmethylthio)-1,2,4-triazole

Following the procedure of Example 1(a)-1(d), except o-tolyl isothiocyanate was substituted for phenylisothiocyanate in step 1(a) and 2-chloromethylthiophene was substituted for benzyl bromide in step 1(d), the title compound was prepared as a white solid. MS (ESI) 303.2 (M+H)⁺.

### Example 8

### Preparation of 3-(2-methyl-anilino)-5-benzylthio-1,2,4-triazole

Following the procedure of Example 1(a)-1(d), except o-tolyl isothiocyanate was substituted for phenylisothiocyanate in step 1(a) the title compound was prepared as a white solid. MS (ESI) 297.2 (M+H)⁺.

### Example 9

### Preparation of 3-(4-chloro-anilino)-5-(thiophen-2-ylmethylthio)-1,2,4-triazole

Following the procedure of Example 1(a)-1(d), except p-chlorophenyl isothiocyanate was substituted for phenylisothiocyanate in step 1(a) and 2-chloromethylthiophene was substituted for benzyl bromide in step 1(d), the title compound was prepared as a white solid. MS (ESI) 322.7 (M)⁺.

### Example 10

### Preparation of 3-(4-methoxy-anilino)-5-(thiophen-2-ylmethylthio)-1,2,4-triazole

Following the procedure of Example 1(a)-1(d), except p-methoxyphenyl isothiocyanate was substituted for phenylisothiocyanate in step 1(a) and 2-chloromethylthiophene was substituted for benzyl bromide in step 1(d), the title compound was prepared as a white solid. MS (ESI) 319.0 (M+H)⁺.

### Example 11

### Preparation of 3-(4-methoxy-anilino)-5-(2-methyl-benzylthio)-1,2,4-triazole

Following the procedure of Example 1(a)-1(d), except p-methoxyphenyl isothiocyanate was substituted for phenylisothiocyanate in step 1(a) and 2-methylbenzyl bromide was substituted for benzyl bromide in step 1(d), the title compound was prepared as a white solid. MS (ESI) 327.2 (M+H)⁺.

### Example 12

### Preparation of 3-(3,4-dimethoxy-anilino)-5-(pyridin-2-ylmethylthio)-1,2,4-triazole

Following the procedure of Example 1(a)-1(d), except 3,4-dimethoxyphenyl isothiocyanate was substituted for phenylisothiocyanate in step 1(a) and 2-(chloromethyl)pyridine was substituted for benzyl bromide in step 1(d), the title compound was prepared as a white solid. MS (ESI) 344.0 (M+H)⁺.

### Example 13

### Preparation of 3-(3,4-dimethoxy-anilino)-5-(3-methoxy-benzylthio)-1,2,4-triazole

Following the procedure of Example 1(a)-1(d), except 3,4-dimethoxyphenyl isothiocyanate was substituted for phenylisothiocyanate in step 1(a) and 3-methoxybenzyl chloride was substituted for benzyl bromide in step 1(d), the title compound was prepared as a white solid. MS (ESI) 373.2 (M+H)⁺.

### Example 14

### Preparation of [5-(2-methyl-thiazol-4-ylmethylthio)-1H-[1,2,4]triazol-3-yl]-pyridin-3-yl-amine

Following the procedure of Example 1(a)-1(d), except 3-pyridyl isothiocyanate was substituted for phenylisothiocyanate in step 1(a) and 4-chloromethyl-2-methylthiazole was substituted for benzyl bromide in step 1(d), the title compound was prepared as a white solid. MS (ESI) 305.2 (M+H)⁺.

### Example 15

### Preparation of [5-(benzylthio)-1H-[1,2,4]triazol-3-yl]-pyridin-3-yl-amine

Following the procedure of Example 1(a)-1(d), except 3-pyridyl isothiocyanate was substituted for phenylisothiocyanate in step 1(a) the title compound was prepared as a white solid. MS (ESI) 284.2 (M+H)⁺.

### Example 16

### Preparation of [5-(3-methoxybenzylthio)-1H-[1,2,4]triazol-3-yl]-pyridin-3-yl-amine

Following the procedure of Example 1(a)-1(d), except 3-pyridyl isothiocyanate was substituted for phenylisothiocyanate in step 1(a) and 3-methoxyphenyl chloride was substituted for benzyl bromide in step 1(d), the title compound was prepared as a white solid. MS (ESI) 314.2 (M+H)⁺.

### Example 17

### Preparation of [5-(2-fluoro-benzylthio)-1H-[1,2,4]triazol-3-y 1-pyridin-3-yl-amine

Following the procedure of Example 1(a)-1(d), except 3-pyridyl isothiocyanate was substituted for phenylisothiocyanate in step 1(a) and 2-fluorobenzyl bromide was substituted for benzyl bromide in step 1(d), the title compound was prepared as a white solid. MS (ESI) 302.2 (M+H)⁺.

### Example 18

### Preparation of [5-(2-methyl-benzylthio)-1H-[1,2,4]triazol-3-yl]-pyridin-3-yl-amine

Following the procedure of Example 1(a)-1(d), except 3-pyridyl isothiocyanate was substituted for phenylisothiocyanate in step 1(a) and 2methylbenzyl bromide was substituted for benzyl bromide in step 1(d), the title compound was prepared as a white solid. MS (ESI) 298.2 (+H)⁺.

### Example 19

### Preparation of [5-(3,4-difluoro-benzylthio)-1H-[1,2,4]triazol-3-yl]-pyridin-3-yl-amine

Following the procedure of Example 1(a)-1(d), except 3-pyridyl isothiocyanate was substituted for phenylisothiocyanate in step 1(a) and 3,4-difluorobenzyl bromide was substituted for benzyl bromide in step 1(d), the title compound was prepared as a white solid. MS (ESI) 320.2 (M+H)⁺.

### Example 20

### Preparation of [5-(2-methoxy-benzylthio)-1H-[1,2,4]triazol-3-yl]-pyridin-3-yl-amine

Following the procedure of Example 1(a)-1(d), except 3-pyridyl isothiocyanate was substituted for phenylisothiocyanate in step 1(a) and 2-methoxybenzyl chloride was substituted for benzyl bromide in step 1(d), the title compound was prepared as a white solid. MS (ESI) 314.2 (M+H)⁺.

### Example 21

### Preparation of 3-(2,4-dimethoxy-anilino)-5-(5-methyl-isoxazol-3-ylmethylthio)-1,2,4-triazole

Following the procedure of Example 1(a)-1(d), except 2,4-dimethoxyphenyl isothiocyanate was substituted for phenylisothiocyanate in step 1(a) and 3-(chloromethyl)-5-methylisoxazole was substituted for benzyl bromide in step 1(d), the title compound was prepared as a white solid. MS (ESI) 348.0 (M+H)⁺.

### Example 22

### Preparation of 3-(2-methyl-4-methoxy-anilino)-5-(2-fluoro-benzylthio)-1,2,4-triazole

Following the procedure of Example 1(a)-1(d), except 2-methyl-4-methoxyphenyl isothiocyanate was substituted for phenylisothiocyanate in step 1(a) and 2-fluorobenzyl bromide was substituted for benzyl bromide in step 1(d), the title compound was prepared as a white solid. MS (ESI) 345.0 (M+H)⁺.

### Example 23

### Preparation of 3-(2-methyl-4-methoxy-anilino)-5-(5-methyl-isoxazol-3-ylmethylthio)-1,2,4-triazole

Following the procedure of Example 1(a)-1(d), except 2-methyl-4-methoxyphenyl isothiocyanate was substituted for phenylisothiocyanate in step 1(a) and 3-(chloromethyl)-5-methylisoxazole was substituted for benzyl bromide in step 1(d), the title compound was prepared as a white solid. MS (ESI) 332.2 (M+H)⁺.

### Example 24

### Preparation of 3-(2-methyl-4-methoxy-anilino)-5-(pyridin-2-ylmethylthio)-1,2,4-triazole

Following the procedure of Example 1(a)-1(d), except 2-methyl-4-methoxyphenyl isothiocyanate was substituted for phenylisothiocyanate in step 1(a) and 2-(chloromethyl)pyridine was substituted for benzyl bromide in step 1(d), the title compound was prepared as a white solid. MS (ESI) 328.2 (M+H)⁺.

### Example 25

### Preparation of 3-(3-methyl-anilino)-5-benzylthio-1,2,4-triazole

Following the procedure of Example 1(a)-1(d), except m-tolyl isothiocyanate was substituted for phenylisothiocyanate in step 1(a), the title compound was prepared as a white solid. MS (ESI) 297.2 (M+H)⁺.

### Example 26

### Preparation of 3-(2,6-dimethyl-anilino)-5-benzylthio-1,2,4-triazole

Following the procedure of Example 1(a)-1(d), except 2,6-dimethylphenyl isothiocyanate was substituted for phenylisothiocyanate in step 1(a) the title compound was prepared as a white solid. MS (ESI) 311.4 (M+H)⁺.

### Example 27

### Preparation of 3-(2,6-dimethyl-anilino)-5-(4-fluoro-benzylthio)-1,2,4-triazole

Following the procedure of Example 1(a)-1(d), except 2,6-dimethylphenyl isothiocyanate was substituted for phenylisothiocyanate in step 1(a) and 4-fluorobenzyl bromide was substituted for benzyl bromide in step 1(d), the title compound was prepared as a white solid. MS (ESI) 329.2 (M+H)⁺.

### Example 28

### Preparation of 3-(2,6-dimethyl-anilino)-(3,4-difluoro-benzylthio)-1,2,4-triazole

Following the procedure of Example 1(a)-1(d), except 2,6-dimethylphenyl isothiocyanate was substituted for phenylisothiocyanate in step 1(a) and 3,4-difluorobenzyl bromide was substituted for benzyl bromide in step 1(d), the title compound was prepared as a white solid. MS (ESI) 347.0 (M+H)⁺.

### Example 29

### Preparation of 3-(2,6-dimethyl-anilino)-5-(2-fluoro-benzylthio)-1,2,4-triazole

Following the procedure of Example 1(a)-1(d), except 2,6-dimethylphenyl isothiocyanate was substituted for phenylisothiocyanate in step 1(a) and 2-fluorobenzyl bromide was substituted for benzyl bromide in step 1(d), the title compound was prepared as a white solid. MS (EST) 329.2 (M+H)⁺.

### Example 30

### Preparation of 3-(2,6-dimethyl-anilino)-5-(2-methyl-benzylthio)-1,2,4-triazole

Following the procedure of Example 1(a)-1(d), except 2,6-dimethylphenyl isothiocyanate was substituted for phenylisothiocyanate in step 1(a) and 2-methylbenzyl bromide was substituted for benzyl bromide in step 1(d), the title compound was prepared as a white solid. MS (ESI) 325.2 (M+H)⁺.

### Example 31

### Preparation of 3-(2,6-dimethyl-anilino)-5-(2-methyl-2-butenylthio)-1,2,4-triazole

Following the procedure of Example 1(a)-1(d), except 2,6-dimethylphenyl isothiocyanate was substituted for phenylisothiocyanate in step 1(a) and 1-bromo-3-methylbut-2-ene was substituted for benzyl bromide in step 1(d), the title compound was prepared as a white solid. MS (ESI) 289.0 (M+H)⁺.

### Example 32

### Preparation of 3-(2-ethyl-anilino)-5-(thiophen-2-ylmethylthio)-1,2,4-triazole

Following the procedure of Example 1(a)-1(d), except 2-ethylphenyl isothiocyanate was substituted for phenylisothiocyanate in step 1(a) and 2-chloromethylthiophene was substituted for benzyl bromide in step 1(d), the title compound was prepared as a white solid. MS (ESI) 317.2 (M+H)⁺.

### Example 33

### Preparation of 3-(2,4-dimethoxy-anilino)-5-(thiophen-2-ylmethylthio)-1,2,4-triazole

Following the procedure of Example 1(a)-1(d), except 2,4-dimethoxyphenyl isothiocyanate was substituted for phenylisothiocyanate in step 1(a) and 2-chloromethylthiophene was substituted for benzyl bromide in step 1(d), the title compound was prepared as a white solid. MS (ESI) 349.0 (M+H)⁺.

### Example 34

### Preparation of [5-(thiophen-2-ylmethylthio)-1H-[1,2,4]triazol-3-yl]-pyridin-3-yl-amine

Following the procedure of Example 1(a)-1(d), except 3-pyridyl isothiocyanate was substituted for phenylisothiocyanate in step 1(a) and 2-chloromethylthiophene was substituted for benzyl bromide in step 1(d), the title compound was prepared as a white solid. MS (ESI) 290.0 (M+H)⁺.

### Example 35

### Preparation of 3-(2-methyl-4-methoxy-anilino)-5-(thiophen-2-ylmethylthio)-1,2,4-triazole

Following the procedure of Example 1(a)-1(d), except 2-methyl-4-methoxyphenyl isothiocyanate was substituted for phenylisothiocyanate in step 1(a) and 2-chloromethylthiophene was substituted for benzyl bromide in step 1(d), the title compound was prepared as a white solid. MS (ESI) 333.2 (M+H)⁺.

### Example 36

### Preparation of 3-(2-phenyl-anilino)-5-(thiophen-2-ylmethylthio)-1,2,4-triazole

Following the procedure of Example 1(a)-1(d), except 2-phenyl-phenyl isothiocyanate was substituted for phenylisothiocyanate in step 1(a) and 2chloromethylthiophene was substituted for benzyl bromide in step 1(d), the title compound was prepared as a white solid. MS (ESI) 365.2 (M+H)⁺.

### Example 37

### Preparation of 3-(2-methoxy-anilino)-5-(furan-2-ylmethylthio)-1,2,4-triazole

Following the procedure of Example 1(a)-1(d), except 2-methoxyphenyl isothiocyanate was substituted for phenylisothiocyanate in step 1(a) and 2-chloromethyl-faran (Berry, J. M.; Watson, C. Y.; Whish, W. J. D.; Threadgill, M. D. J. Chem. Soc. Perkin Trans. 1 1997, 8, 1147) was substituted for benzyl bromide in step 1(d), the title compound was prepared as a white solid. MS (ESI) 303.2 (M+H)⁺.

### Example 38

### Preparation of 3-(2-methoxy-anilino)-5-(5-chloro-thiophen-2-ylmethylthio)-1,2,4-triazole

Following the procedure of Example 1(a)-1(d), except 2-methoxyphenyl isothiocyanate was substituted for phenylisothiocyanate in step 1(a) and 2-chloromethyl-5-chloro-thiophene was substituted for benzyl bromide in step 1(d), the title compound was prepared as a white solid. MS (FSI) 353.0 (M+H)⁺.

### Example 39

### Preparation of 3-anilino-5-(5-methyl-thiophen-2-ylmethylthio)-1,2,4-triazole

Following the procedure of Example 1(a)-1(d), except 2-chloromethyl-5-methyl-thiophene (Moradpour, A. J. Chem. Soc. Perkin Trans. 1, 1993, 1, 7) was substituted for benzyl bromide in step 1(d), the title compound was prepared as a white solid. MS (ESI) 303.2 (M+H)⁺.

### Example 40

### Preparation of 3-anilino-5-(5-chloro-thiophen-2-ylmethylthio)-1,2,4-triazole

Following the procedure of Example 1(a)-1(d), except 2-chloromethyl-5-chloro-thiophene was substituted for benzyl bromide in step 1(d), the title compound was prepared as a white solid. MS (ESI) 323.0 (M+H)⁺.

### Example 41

### Preparation of 3-(2-methyl-anilino)-5-(5-bromo-thiophen-2-ylmethylthio)-1,2,4-triazole

Following the procedure of Example 1(a)-1(d), except o-tolyl isothiocyanate was substituted for phenylisothiocyanate in step 1(a) and 2-chloromethyl-5-bromo-thiophene (Clapp, R. C.; Clark, J. H; Vaughan, J. R.; English, J. P.; Anderson, G. W. J. Am. Chem. Soc. 1947, 60, 1549) was substituted for benzyl bromide in step 1(d), the title compound was prepared as a white solid. MS (ESI) 381.0 (M)⁺.

### Example 42

### Preparation of 3-(3-methyl-anilino)-5-(thiophen-2-ylmethylthio)-1,2,4-triazole

Following the procedure of Example 1(a)-1(d), except m-tolyl isothiocyanate was substituted for phenylisothiocyanate in step 1(a) and 2-chloromethylthiophene was substituted for benzyl bromide in step 1(d), the title compound was prepared as a white solid. MS (ESI) 303.2 (M+H)⁺.

### Example 43

### Preparation of 3-(3-methyl-anilino)-5-(3-chloro-thiophen-2-ylmethylthio)-1,2,4-triazole

Following the procedure of Example 1(a)-1(d), except 3-m-tolyl isothiocyanate was substituted for phenylisothiocyanate in step 1(a) and 2-chloromethyl-3-chloro-thiophene (Chauhan, P. M. S.; Jenkins, G.; Walker, S. M.; Storr, R. C. Tetrahedron Lett. 1988, 29(1), 117) was substituted for benzyl bromide in step 1(d), the title compound was prepared as a white solid. MS (ESI) 337.2 (M+H)⁺.

### Example 44

### Preparation of 3-(sec-butyl-anilino)-5-(furan-2-ylmethylmethylthio)-1,2,4-triazole

Following the procedure of Example 1(a)-1(d) except sec-butyl-phenyl isothiocyanate was substituted for phenylisothiocyanate in step 1(a) and 2-chloromethyl-furan (Berry, J. M.; Watson, C. Y.; Whish, W. J. D.; Threadgill, M. D. J. Chem. Soc. Perkin Trans. 1 1997, 8, 1147) was substituted for benzyl bromide in step 1(d), the title compound was prepared as a white solid. MS (ESI) 329.2 (M+H)⁺.

### Example 45

### Preparation of 3-(3-methoxy-anilino)-5-(thiophen-2-ylmethylthio)-1,2,4-triazole

Following the procedure of Example 1(a)-1(d), except 3-methoxy-phenyl isothiocyanate was substituted for phenylisothiocyanate in step 1(a) and 2-chloromethylthiophene was substituted for benzyl bromide in step 1(d), the title compound was prepared as a white solid. MS (ESI) 319.2 (M+H)⁺.

### Example 46

### Preparation of 3-(3-methoxy-anilino)-5-(furan-2-ylmethylthio)-1,2,4-triazole

Following the procedure of Example 1(a)-1(d), except 3-methoxy-phenyl isothiocyanate was substituted for phenylisothiocyanate in step 1(a) and 2-chloromethyl-furan (Berry, J. M.; Watson, C. Y.; Whish, W. J. D.; Threadgill, M. D. J. Chem. Soc. Perkin Trans. 1 1997, 8, 1147) was substituted for benzyl bromide in step 1(d), the title compound was prepared as a white solid. MS (ESI) 303.2 (M+H)⁺.

### Example 47

### Preparation of 3-(4-methoxy-anilino)-5-(furan-2-ylmethylthio)-1,2,4-triazole

Following the procedure of Example 1(a)-1(d), except p-methoxy-phenyl isothiocyanate was substituted for phenylisothiocyanate in step 1(a) and 2-chloromethyl-furan (Berry, J. M.; Watson, C. Y.; Whish, W. J. D.; Threadgill, M. D. J. Chem. Soc. Perkin Trans. 1 1997, 8, 1147) was substituted for benzyl bromide in step 1(d), the title compound was prepared as a white solid. MS (ESI) 303.2 (M+H)⁺.

### Example 48

### Preparation of 3-(5-Benzyl-1H-[1,2,4]-triazole-3-yl sulfanyl)-propionic acid methyl ester

Following the procedure of Example 1(a)-1(d), except 3-bromo-propionic acid methyl ester was substituted for benzyl bromide in step 1(d), the title compound was prepared as a white solid. MS (ESI) 278.3 (M+H)⁺.

### Example 49

### Preparation of 3-(4-Hydroxy-anilino)-5-benzylthio-1,2,4-triazole

### a. 3-(4-Methoxy-anilino)-5-benzylthio-1,2,4-triazole

Following the procedure of Example 1(a)-1(d), except p-methoxyphenyl isothiocyanate was substituted for phenylisothiocyanate in step 1(a), the title compound was prepared as a white solid. MS (ESI) 313.2 (M+H)⁺.

### b. 3-(4-Hydroxy-anilino)-5-benzylthio-1,2,4-triazole

A solution of 3-(4-methoxy-anilino)-5-benzylthio-1,2,4-triazole (300 mg, 0.96 mmol) in conc. aqueous HBr (15 ml) was heated at reflux for 24 h. The HBr was removed under reduced pressure and the crude product was purified by preparative HPLC to afford the title compound as a yellow solid. MS (ESI) 299.2 (M+H)⁺.

### Example 50

### Preparation of 3-(2-Hydroxy-anilino)-5-benzylthio-1,2,4-triazole

Following the procedure of Example 48(a)-1(b), except o-methoxyphenyl isothiocyanate was substituted for p-methoxyphenyl isothiocyanate in step 48(a), the title compound was prepared as a white solid. MS (ESI) 299.2 (M+H)⁺.

### Example 51

### Preparation of 3-(3-methyl-anilino)-5-(furan-2-ylmethylthio)-1,2,4-triazole

Following the procedure of Example 1(a)-1(d), except 3-m-tolyl isothiocyanate was substituted for phenylisothiocyanate in step 1(a) and 2-chloromethyl-furan (Berry, J. M.; Watson, C. Y.; Whish, W. J. D.; Threadgill, M. D. J. Chem. Soc. Perkin Trans. 1 1997, 8, 1147) was substituted for benzyl bromide in step 1(d), the title compound was prepared as a white solid. MS (ESI) 287.2 (M+H)⁺.

Biological Data:

Coupled Spectrophotometric Assays of MetAP:

MetAP activity can be measured spectrophotometrically by monitoring the free L-amino acid formation. The release of N-terminal methionine from a tripeptide (Met-Ala-Ser, Sigma) or a tetrapeptide (Met-Gly-Met-Met, Sigma) substrate was assayed using the L-amino acid oxidase (AAO)/horse radish peroxidase (HRP) couple (eq. 1-3a,b). The formation of hydrogen peroxide (H₂O₂) was continuously monitored at 450 nm (absorbance increase of o-Dianisidine (Sigma) upon oxidation, Δε=15,300 M⁻¹cm⁻¹)² and 30° C. in a 96- or 384-well plate reader by a method adapted from Tsunasawa, S. et al. (1997) (eq. 3a). Alternatively, formation of H₂O₂ was followed by monitoring the fluorescence emission increase at 587 nm (Δε=54,000 M⁻¹cm⁻¹, λₑₓ=563 nm, slit width for both excitation and emission was 1.25 nm) and 30° C. using Amplex Red (Molecular Probes, Inc) (Zhou, M. et al. (1997) Anal. Biochem. 253, 162) (eq. 3b). In a total volume of 50 uL, a typical assay contained 50 mM Hepes-Na⁺, pH 7.5, 100 mM NaCl, 10 uM CoCl₂, 1 mM O-Dianisidine or 50 uM Amplex Red, 0.5 units of HRP (Sigma), 0.035 unit of AAO (Sigma), 1 nM MetAP, and varying amounts of peptide substrates. Assays were initiated by the addition of MetAP enzyme, and the rates were corrected for the background rate determined in the absence of MetAP. 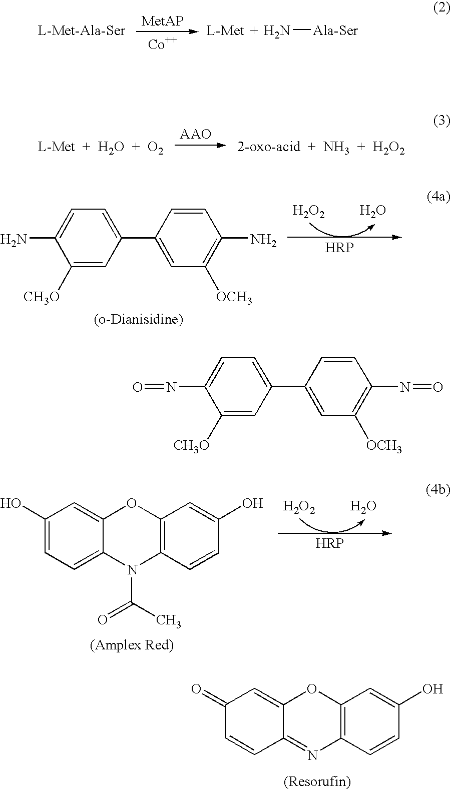
Kinetic Data Analysis:

Data were fitted to the appropriate rate equations using Grafit computer software. Initial velocity data conforming to Michaelis-Menton kinetics were fitted to eq. 4. Inhibition patterns conforming to apparent competitive and non-competitive inhibition were fitted to eq. 5 and eq. 6, respectively.
ν=VA/(Kₐ+A) (4)
ν=VA/[Kₐ(1+I/Kᵢₛ)+A] (5)
ν=VA/[Kₐ(1+I/Kᵢₛ)+A(1+I/Kᵢᵢ)] (6)
In eqs. 4-6, ν is the initial velocity, V is the maximum velocity, Kₐ is the apparent Michaelis constant, I is the inhibitor concentration, and A is the concentration of variable substrates. The nomenclature used in the rate equations for inhibition constants is that of Cleland (1963), in which Kᵢₛ and Kᵢᵢ represent the apparent slope and intercept inhibition constants, respectively.
Antimicrobial Activity Assay

Whole-cell antimicrobial activity was determined by broth microdilution using the National Committee for Clinical Laboratory Standards (NCCLS) recommended procedure, Document M7-A4, “Methods for Dilution Susceptibility Tests for Bacteria that Grow Aerobically” (incorporated by reference herein). The compound was tested in serial two-fold dilutions ranging from 0.06 to 64 mcg/ml. A panel of 12 strains were evaluated in the assay. This panel consisted of the following laboratory strains: Staphylococcus aureus Oxford, Streptococcus pneumoniae R6, Streptococcus pyogenes CN10, Enterococcus faecalis I, Haemophilus influenzae Q1, Escherichia coli DC0, E. coli EES, E. coli 7623 (AcrAB+) E. coli 120 (AcrAB-) Klebsiella pneumoniae E70, Pseudomonas aeruginosa K799 wt and Candida albicans GR1681. The minimum inhibitory concentration (MIC) was determined as the lowest concentration of compound that inhibited visible growth. A mirror reader was used to assist in determining the MIC endpoint.

The compounds of this invention show MetAP inhibitor activity having IC₅₀ values in the range of 0.0001 to 100 uM. The full structure/activity relationship has not yet been established for the compounds of this invention. However, given the disclosure herein, one of ordinary skill in the art can utilize the present assays in order to determine which compounds of this invention are inhibitors of MetAP and which bind thereto with an IC₅₀ value in the range of 0.0001 to 100 uM.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

1. A method of treating bacterial infections by administering a compound of formula (IA), or a pharmaceutically acceptable salt or solvate thereof 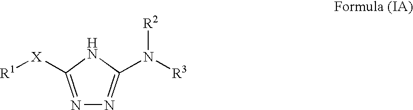
wherein,
X is S or O;
R¹ is optionally substituted C₁₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl;
R² is optionally substituted C₂₋₆alkyl, C₃₋₆alkenyl, C₃₋₆alkynyl, optionally substituted Ar—C₀₋₆alkyl, optionally substituted Het-C₀₋₆alkyl, C₃₋₇cycloalkyl-C₀₋₆alkyl;
R³ is H, optionally substituted C₁₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl, C₀₋₆alkyl-C(O)X′AB, C₀₋₆alkyl-S(O)₂X′AB, C₀₋₆alkyl-X′AB, wherein X′ is O, S, C or N; A and B are independently H, optionally substituted C₁₋₆alkyl, C₃₋₆alkenyl, C₃₋₆alkynyl, optionally substituted Ar—C₀₋₆alkyl, optionally substituted Het-C₀₋₆alkyl, C₃₋₇cycloalkyl-C₀₋₆alkyl, or A or B are independently absent.

2. The method of claim 1, wherein the compound of formula (IA) is selected from:
3-anilino-5-benzylthio-1,2,4-triazole;
3-anilino-5-(thiophen-2-ylmethylthio)-1,2,4-triazole;
3-anilino-5-(3,5-dimethyl-benzylthio)-1,2,4-triazole;
3-(4-methyl-anilino)-5-(pyridin-4-ylmethylthio)-1,2,4-triazole;
3-(2-methyl-anilino)-5-benzylthio-1,2,4-triazole;
3-(4-methyl-anilino)-5-(pyridin-2-ylmethylthio)-1,2,4-triazole;
3-(4-chloro-anilino)-5-(thiophen-2-ylmethylthio)-1,2,4-triazole;
3-(2-methyl-anilino)-5-(thiophen-2-ylmethylthio)-1,2,4-triazole;
3-(4-methoxy-anilino)-5-(thiophen-2-ylmethylthio)-1,2,4-triazole;
3-(4-methoxy-anilino)-5-(pyridin-4-ylmethylthio)-1,2,4-triazole;
3-(4-methoxy-anilino)-5-(2-methyl-benzylthio)-1,2,4-triazole;
3-(3,4-dimethoxy-anilino)-5-(3-methoxy-benzylthio)-1,2,4-triazole;
3-(3,4-dimethoxy-anilino)-5-(pyridin-2-ylmethylthio)-1,2,4-triazole;
[5-(benzylthio)-1H-[1,2,4]triazol-3-yl]-pyridin-3-yl-amine;
[5-(3-methoxybenzylthio)-1H-[1,2,4]triazol-3-yl]-pyridin-3-yl-amine;
[5-(2-fluoro-benzylthio)-1H-[1,2,4]triazol-3-yl]-pyridin-3-yl-amine;
[5-(2-methyl-benzylthio)-1H-[1,2,4]triazol-3-yl]-pyridin-3-yl-amine;
[5-(3,4-difluoro-benzylthio)-1H-[1,2,4]triazol-3-yl]-pyridin-3-yl-amine;
[5-(2-methoxy-benzylthio)-1H-[1,2,4]triazol-3-yl]-pyridin-3-yl-amine;
[5-(2-methyl-thiazol-4-ylmethylthio)-1H-[1,2,4]triazol-3-yl]-pyridin-3-yl-amine;
3-(2,4-dimethoxy-anilino)-5-(5-methyl-isoxazol-3-ylmethylthio)-1,2,4-triazole;
3-(2-methyl-4-methoxy-anilino)-5-(2-fluoro-benzylthio)-1,2,4-triazole;
3-(2-methyl-4-methoxy-anilino)-5-(5-methyl-isoxazol-3-ylmethylthio)-1,2,4-triazole;
3-(2-methyl-4-methoxy-anilino)-5-(pyridin-2-ylmethylthio)-1,2,4-triazole;
3-(3-methyl-anilino)-5-benzylthio-1,2,4-triazole;
3-(2,6-dimethyl-anilino)-5-(4-fluoro-benzylthio)-1,2,4-triazole;
3-(2,6-dimethyl-anilino)-5-(3,4-difluoro-benzylthio)-1,2,4-triazole;
3-(2,6-dimethyl-anilino)-5-(2-methyl-2-butenylthio)-1,2,4-triazole;
3-(2,6-dimethyl-anilino)-5-(2-fluoro-benzylthio)-1,2,4-triazole;
3-(2,6-dimethyl-anilino)-5-benzylthio-1,2,4-triazole;
3-(2,6-dimethyl-anilino)-5-(2-methyl-benzylthio)-1,2,4-triazole;
3-(2-ethyl-anilino)-5-(thiophen-2-ylmethylthio)-1,2,4-triazole;
3-(3-methyl-anilino)-5-(thiophen-2-ylmethylthio)-1,2,4-triazole;
3-(2-phenyl-anilino)-5-(thiophen-2-ylmethylthio)-1,2,4-triazole;
3-(2,4-dimethoxy-anilino)-5-(thiophen-2-ylmethylthio)-1,2,4-triazole;
[5-(thiophen-2-ylthio)-1H-[1,2,4]triazol-3-yl]-pyridin-3-yl-amine;
3-(2-methyl-4-methoxy-anilino)-5-(thiophen-2-ylmethylthio)-1,2,4-triazole;
3-(4-hydroxy-anilino)-5-benzylthio-1,2,4-triazole;
3-(2-methoxy-anilino)-5-(furan-2-ylmethylthio)-1,2,4-triazole;
3-anilino-5-(5-methyl-thiophen-2-ylmethylthio)-1,2,4-triazole;
3-(2-methyl-anilino)-5-(5-bromo-thiophen-2-ylmethylthio)-1,2,4-triazole;
3-(2-methoxy-anilino)-5-(5-chloro-thiophen-2-ylmethylthio)-1,2,4-triazole;
3-(3-methyl-anilino)-5-(3-chloro-thiophen-2-ylmethylthio)-1,2,4-triazole;
3-anilino-5-(5-chloro-thiophen-2-ylmethylthio)-1,2,4-triazole;
3-(3-methyl-anilino)-5-(furan-2-ylmethylthio)-1,2,4-triazole;
3-(2-Hydroxy-anilino)-5-benzylthio-1,2,4-triazole;
3-(3-methoxy-anilino)-5-(thiophen-2-ylmethylthio)-1,2,4-triazole;
3-(sec-butyl-anilino)-5-(furan-2-ylmethylthio)-1,2,4-triazole;
3-(3-methoxy-anilino)-5-(furan-2-ylmethylthio)-1,2,4-triazole;
3-(4-methoxy-anilino)-5-(furan-2-ylmethylthio)-1,2,4-triazole; and
3-(5-Benzyl-1H-[1,2,4]-triazole-3-yl sulfanyl)-propionic acid methyl ester,
or a pharmaceutically acceptable salt or solvate thereof.

3. A method for treating a bacterial infection mediated by MetAP in mammals, comprising administering to a mammal in need of such treatment, an effective amount of a compound of formula (IA) or a pharmaceutically acceptable salt or solvate thereof: 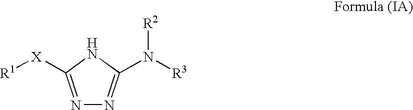
wherein,
X is S or O;
R¹ is optionally substituted C₁₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl;
R² is optionally substituted C₂₋₆alkyl, C₃₋₆alkenyl, C₃₋₆alkynyl, optionally substituted Ar—C₀₋₆alkyl, optionally substituted Het-C₀₋₆alkyl, C₃₋₇cycloalkyl-C₀₋₆alkyl;
R³ is H, optionally substituted C₁₋₆alkyl, C₃₋₆alkenyl, C₃₋₆alkynyl, optionally substituted Ar—C₀₋₆alkyl, optionally substituted Het-C₀₋₆alkyl, or C₃₋₇cycloalkyl-C₀₋₆alkyl, C₀₋₆alkyl-C(O)X′AB, C₀₋₆alkyl-S(O)₂X′AB, C₀₋₆alkyl-X′AB, wherein X′ is O, S, C or N; A and B are independently H, optionally substituted C₁₋₆alkyl, C₃₋₆alkenyl, C₃₋₆alkynyl, optionally substituted Ar—C₀₋₆alkyl, optionally substituted Het-C₀₋₆alkyl, C₃₋₇cycloalkyl-C₀₋₆alkyl, or A or B are independently absent.

4. The method of claim 3 wherein, the compound of formula (IA) is selected from:
3-anilino-5-benzylthio-1,2,4-triazole;
3-anilino-5-(thiophen-2-ylmethylthio)-1,2,4-triazole;
3-anilino-5-(3,5-dimethyl-benzylthio)-1,2,4-triazole;
3-(4-methyl-anilino)-5-(pyridin-4-ylmethylthio)-1,2,4-triazole;
3-(2-methyl-anilino)-5-benzylthio-1,2,4-triazole;
3-(4-methyl-anilino)-5-(pyridin-2-ylmethylthio)-1,2,4-triazole;
3-(4-chloro-anilino)-5-(thiophen-2-ylmethylthio)-1,2,4-triazole;
3-(2-methyl-anilino)-5-(thiophen-2-ylmethylthio)-1,2,4-triazole;
3-(4-methoxy-anilino)-5-(thiophen-2-ylmethylthio)-1,2,4-triazole;
3-(4-methoxy-anilino)-5-(pyridin-4-ylmethylthio)-1,2,4-triazole;
3-(4-methoxy-anilino)-5-(2-methyl-benzylthio)-1,2,4-triazole;
3-(3,4-dimethoxy-anilino)-5-(3-methoxy-benzylthio)-1,2,4-triazole;
3-(3,4-dimethoxy-anilino)-5-(pyridin-2-ylmethylthio)-1,2,4-triazole;
[5-(benzylthio)-1H-(1,2,4]triazol-3-yl]-pyridin-3-yl-amine;
[5-(3-methoxybenzylthio)-1H-[1,2,4]triazol-3-yl]-pyridin-3-yl-amine;
[5-(2-fluoro-benzylthio)-1H-[1,2,4]triazol-3-yl]-pyridin-3-yl-amine;
[5-(2-methyl-benzylthio)-1H-[1,2,4]triazol-3-yl]-pyridin-3-yl-amine;
[5-(3,4-difluoro-benzylthio)-1H-[1,2,4]triazol-3-yl]-pyridin-3-yl-amine;
[5-(2-methoxy-benzylthio)-1H-[1,2,4]triazol-3-yl]-pyridin-3-yl-amine;
[5-(2-methyl-thiazol-4-ylmethylthio)-1H-[1,2,4]triazol-3-yl]-pyridin-3-yl-amine;
3-(2,4-dimethoxy-anilino)-5-(5-methyl-isoxazol-3-ylmethylthio)-1,2,4-triazole;
3-(2-methyl-4-methoxy-anilino)-5-(2-fluoro-benzylthio)-1,2,4-triazole;
3-(2-methyl-4-methoxy-anilino)-5-(5-methyl-isoxazol-3-ylmethylthio)-1,2,4-triazole;
3-(2-methyl-4-methoxy-anilino)-5-(pyridin-2-ylmethylthio)-1,2,4-triazole;
3-(3-methyl-anilino)-5-benzylthio-1,2,4-triazole;
3-(2,6-dimethyl-anilino)-5-(4-fluoro-benzylthio)-1,2,4-triazole;
3-(2,6-dimethyl-anilino)-5-(3,4-difluoro-benzylthio)-1,2,4-triazole;
3-(2,6-dimethyl-anilino)-5-(2-methyl-2-butenylthio)-1,2,4-triazole;
3-(2,6-dimethyl-anilino)-5-(2-fluoro-benzylthio)-1,2,4-triazole;
3-(2,6-dimethyl-anilino)-5-benzylthio-1,2,4-triazole;
3-(2,6-dimethyl-anilino)-5-(2-methyl-benzylthio)-1,2,4-triazole;
3-(2-ethyl-anilino)-5-(thiophen-2-ylmethylthio)-1,2,4-triazole;
3-(3-methyl-anilino)-5-(thiophen-2-ylmethylthio)-1,2,4-triazole;
3-(2-phenyl-anilino)-5-(thiophen-2-ylmethylthio)-1,2,4-triazole;
3-(2,4-dimethoxy-anilino)-5-(thiophen-2-ylmethylthio)-1,2,4-triazole;
[5-(thiophen-2-ylthio)-1H-[1,2,4]triazol-3-yl]-pyridin-3-yl-amine;
3-(2-methyl-4-methoxy-anilino)-5-(thiophen-2-ylmethylthio)-1,2,4-triazole;
3-(4-hydroxy-anilino)-5-benzylthio-1,2,4-triazole;
3-(2-methoxy-anilino)-5-(furan-2-ylmethylthio)-1,2,4-triazole;
3-anilino-5-(5-methyl-thiophen-2-ylmethylthio)-1,2,4-triazole;
3-(2-methyl-anilino)-5-(5-bromo-thiophen-2-ylmethylthio)-1,2,4-triazole;
3-(2-methoxy-anilino)-5-(5-chloro-thiophen-2-ylmethylthio)-1,2,4-triazole;
3-(3-methyl-anilino)-5-(3-chloro-thiophen-2-ylmethylthio)-1,2,4-triazole;
3-anilino-5-(5-chloro-thiophen-2-ylmethylthio)-1,2,4-triazole;
3-(3-methyl-anilino)-5-(furan-2-ylmethylthio)-1,2,4-triazole;
3-(2-Hydroxy-anilino)-5-benzylthio-1,2,4-triazole;
3-(3-methoxy-anilino)-5-(thiophen-2-ylmethylthio)-1,2,4-triazole;
3-(sec-butyl-anilino)-5-(furan-2-ylmethylthio)-1,2,4-triazole;
3-(3-methoxy-anilino)-5-(furan-2-ylmethylthio)-1,2,4-triazole;
3-(4-methoxy-anilino)-5-(furan-2-ylmethylthio)-1,2,4-triazole; and
3-(5-Benzyl-1H-[1,2,4]-triazole-3-yl sulfanyl)-propionic acid methyl ester,
or a pharmaceutically acceptable salt or solvate thereof.
